(19) United States
(12) Patent Application Publication
(10) Pub. No.: US 2021/0023331 A1
(43) Pub. Date: Jan. 28, 2021

(54) COMPUTER ARCHITECTURE FOR IDENTIFYING SLEEP STAGES

(71) Applicant: Regents of the University of Minnesota, Minneapolis, MN (US)

(72) Inventors: Karan Aggarwal, Seattle, WA (US); Swaraj Khadanga, San Jose, CA (US); Shafiq Rayhan Joty, Singapore (SG); Louis Kazaglis, Bentleyville, OH (US); Jaideep Srivastava, Plymouth, MN (US)

(21) Appl. No.: 16/932,348

(22) Filed: Jul. 17, 2020

Related U.S. Application Data
(60) Provisional application No. 62/877,134, filed on Jul. 22, 2019.

(51) Int. Cl. A61M 21/02; A61B 5/087; A61B 5/00; A61M 16/00; G16H 40/67; G06N 3/04; F24F 11/63

(52) U.S. Cl. CPC A61M 21/02; A61B 5/087; A61B 5/4812; A61B 5/7264; A61B 5/6803; A61M 2021/0027; A61M 16/024; G16H 40/67; G06N 3/04; F24F 11/63; A61B 5/0002

(57) ABSTRACT

A computing machine receives sensor data representing airflow or air pressure. The computing machine determines, using an artificial neural network, a current sleep stage corresponding to the sensor data. The current sleep stage is one of: wake, rapid eye movement (REM), light sleep, and deep sleep. The artificial neural network comprises a convolutional neural network (CNN), a recurrent neural network (RNN), and a conditional random field (CRF). The computing machine provides an output representing the current sleep stage.

### PRIORITY CLAIM

This application claims priority to US Provisional Patent Application No. 62/877,134, filed on Jul. 22, 2019, entitled “SLEEP STAGING FOR MONITORING SLEEP APNEA PATIENTS ON CPAP THERAPY,” the entire content of which is incorporated herein by reference,

### TECHNICAL FIELD

Embodiments pertain to computer architecture. Some embodiments relate to neural networks. Some embodiments relate to using neural networks in identifying sleep stages of a person.

### BACKGROUND

Sleep apnea is a medical condition involving airway collapse during sleep resulting in reduced oxygen supply to the brain and patient to wake, Sleep apnea is most commonly treated with Continuous Positive Air Pressure (CPAP) therapy. CPAP is an in-home therapy where patients wear a mask with adaptive pressure during the sleep. Presently, however, there is no mechanism to monitor a patient's progress with CPAP.

### BRIEF DESCRIPTION OF THE DRAWINGS

FIG. 1 illustrates the training and use of a machine-learning program, in accordance with some embodiments.

FIG. 2 illustrates an example neural network, in accordance with some embodiments.

FIG. 3 illustrates the training of an image recognition machine learning program, in accordance with some embodiments.

FIG. 4 illustrates the feature-extraction process and classifier training, in accordance with some embodiments.

FIG. 5 is a block diagram of a computing machine, in accordance with some embodiments.

FIG. 6 illustrates an example use case of a Continuous Positive Air Pressure (CPAP) device, in accordance with some embodiments.

FIG. 7 illustrates an example sleep state transition diagram, in accordance with some embodiments.

FIG. 8 illustrates an example artificial neural network that may be used in sleep stage modeling, in accordance with some embodiments.

FIG. 9 illustrates an example of flow signal and corresponding sleep stage annotations, in accordance with some embodiments.

FIG. 10A illustrates an example next stage/previous stage table for a conditional random field (CRF) sleep stage transition matrix, in accordance with sonic embodiments.

FIG. 10B illustrates an example predicted stage/true stage table for a neural CRF model, in accordance with some embodiments.

FIG. 10C illustrates an example predicted stage/true stage table for a cost-sensitive neural CRF model, in accordance with some embodiments.

FIG. 11 is a flow chart of an example method for identifying sleep stages, in accordance with some embodiments.

### DETAILED DESCRIPTION

The following description and the drawings sufficiently illustrate. specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

Sleep apnea is a medical condition involving airway collapse during sleep resulting in reduced oxygen supply to the brain and patient to wake. Sleep apnea is most commonly treated with Continuous Positive Air Pressure (CPAP) therapy. CPAP is an in-home therapy where patients wear a mask with adaptive pressure during the sleep. Presently, however, there is no mechanism to monitor a patient's progress with CPAP, though doctors get real-time pressure-flow data. Accurate sleep stages from CPAP is useful for such a mechanism. Some aspects are directed to an automated sleep staging model based the flow signal. Some aspects include an end-to-end framework that uses a combination of deep neural networks to extract high-level features from raw signals with a structured output layer based on a conditional random field to model the temporal transition structure of the sleep stages. The disclosed technique, in some aspects, can be used to accurately track the response of sleep apnea patients on CPAP therapy, where no such automated mechanism exists. Health-care providers can monitor the patients from convenience of the patient's home, allowing for personalized early interventions for CPAP therapy, which presently, in some cases, suffers from abandonment issues.

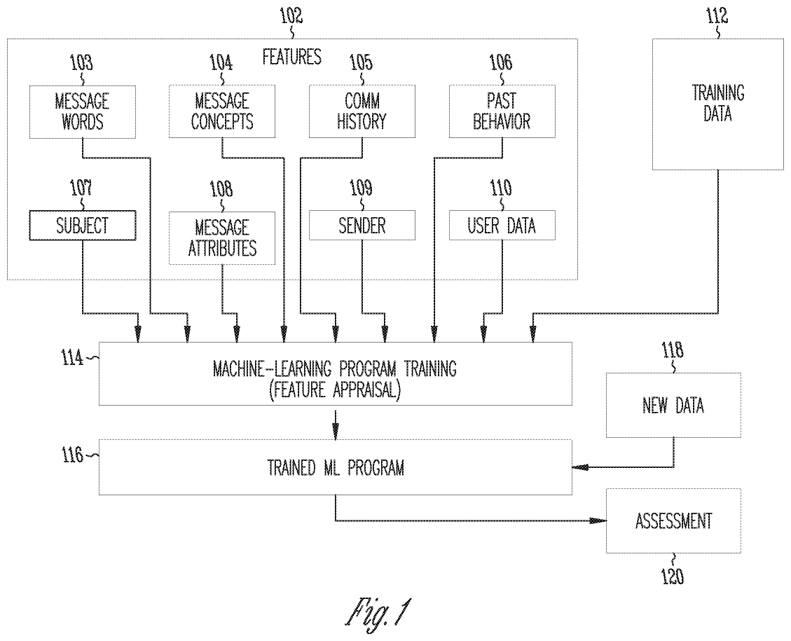

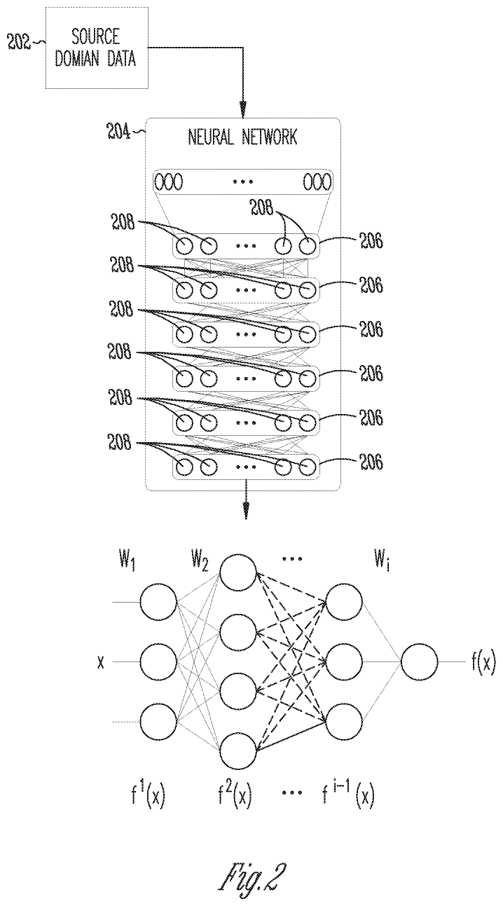

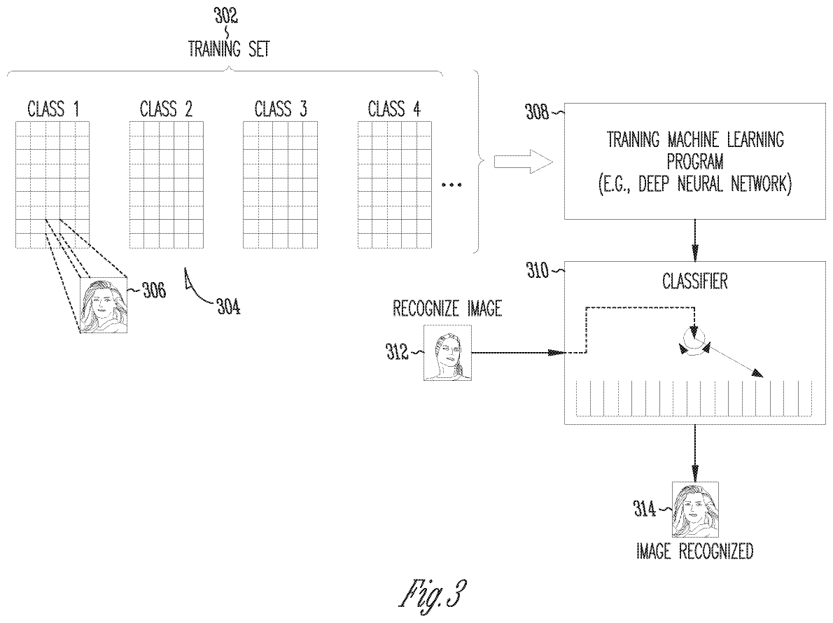

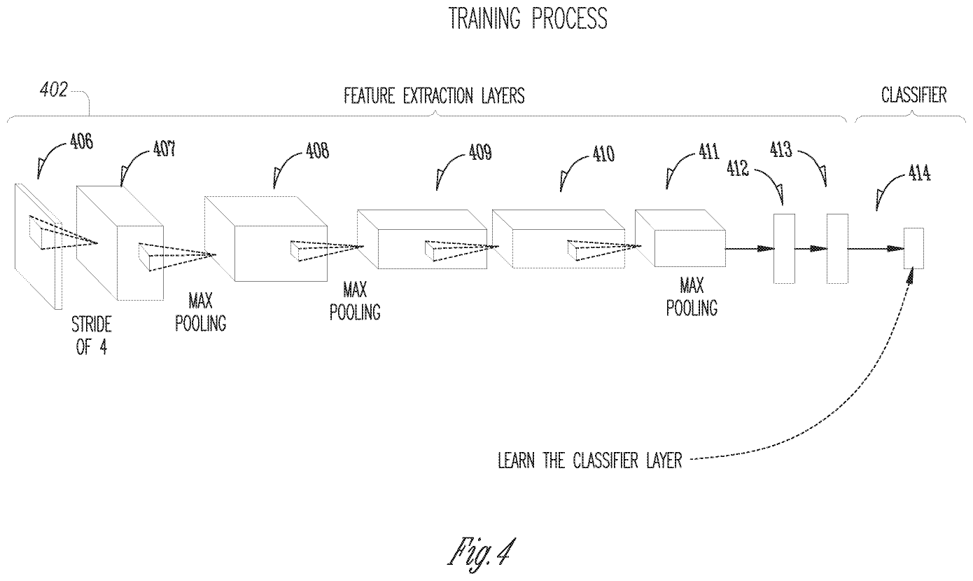

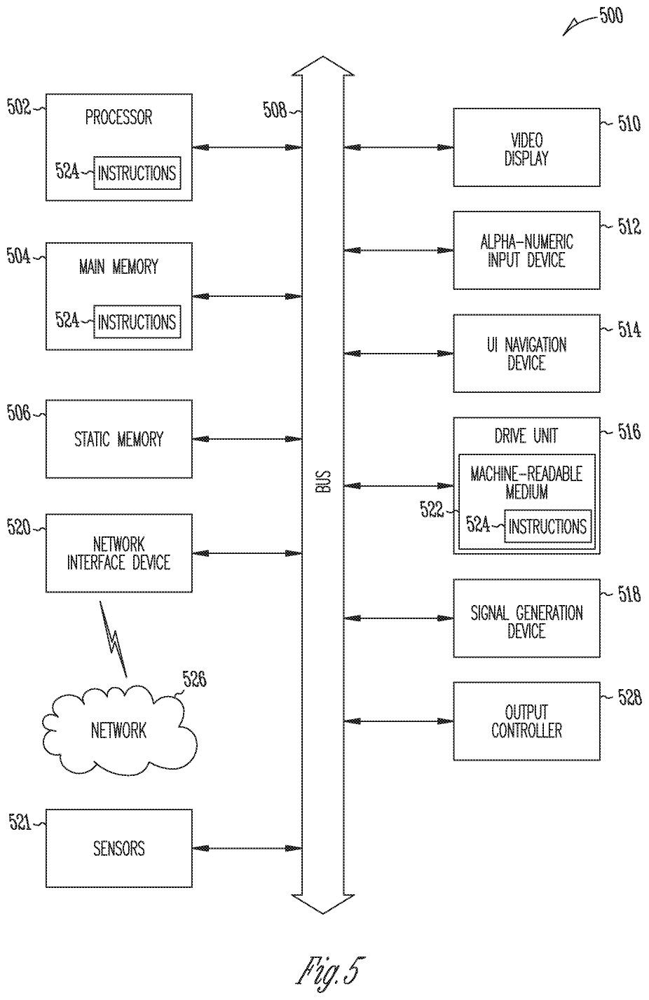

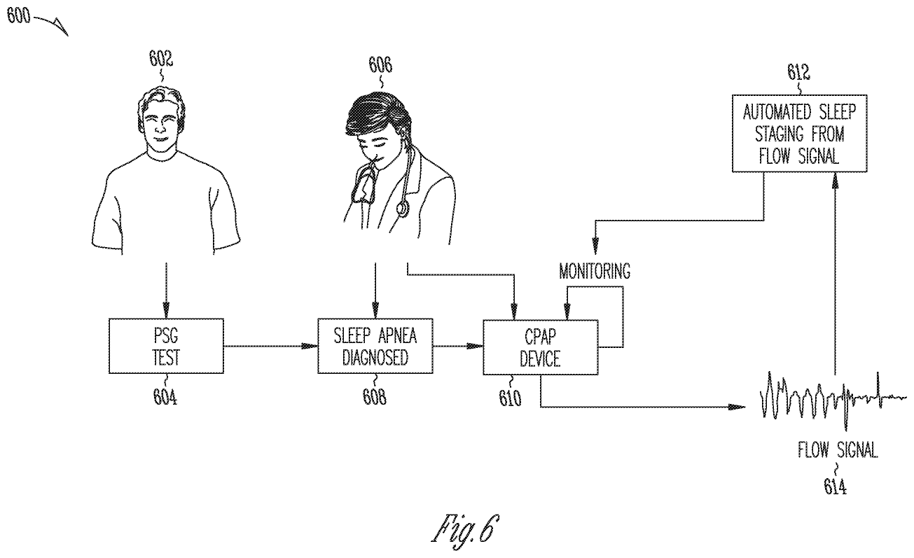

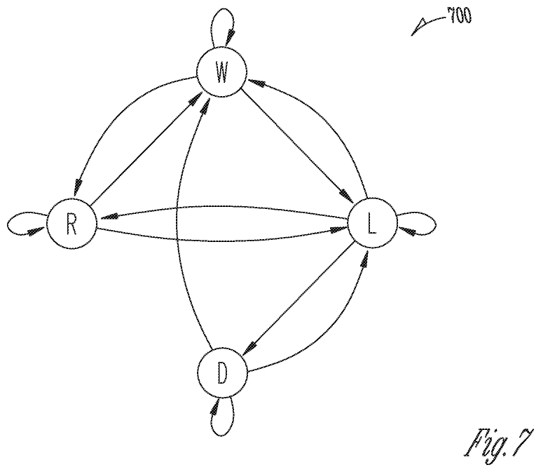

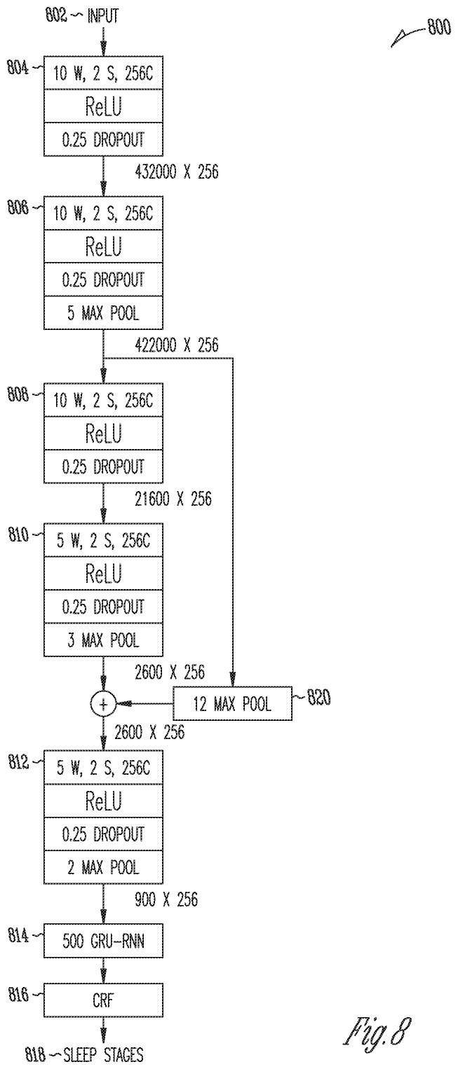

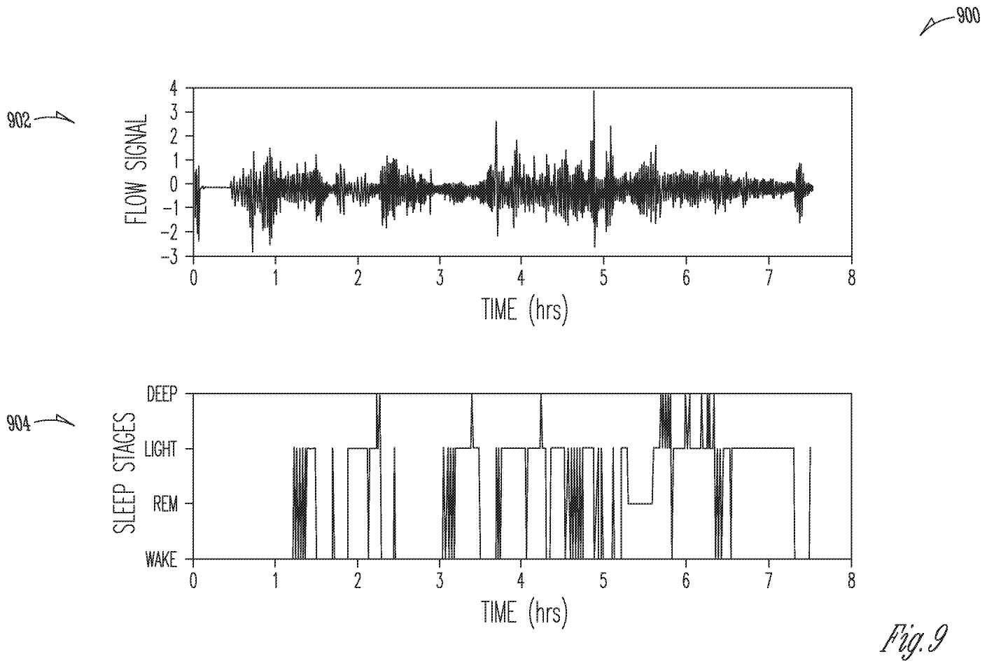

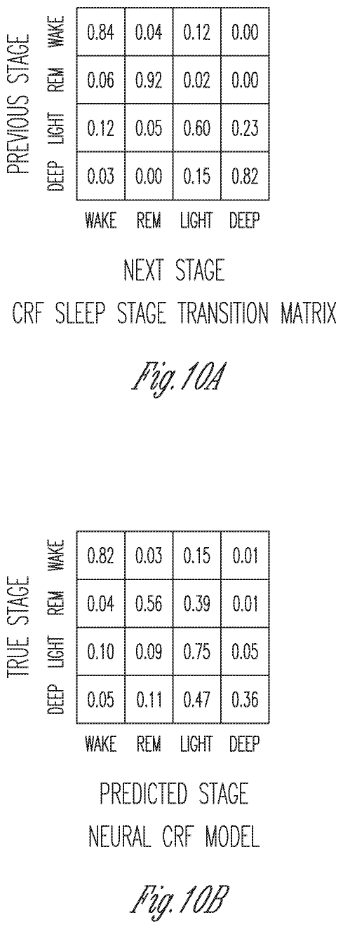

FIG. 1 illustrates the training and use of a machine-learning program, according to some example embodiments. In some example embodiments, machine-learning programs (MLPs), also referred to as machine-learning algorithms or tools, are utilized to perform operations associated with machine learning tasks, such as image recognition or machine translation.

Machine learning is a field of study that gives computers the ability to learn without being explicitly programmed. Machine learning explores the study and construction of algorithms, also referred to herein as tools, which may learn from existing data and make predictions about new data. Such machine-learning tools operate by building a model from example training data 112 in order to make data-driven predictions or decisions expressed as outputs or assessments 120. Although example embodiments are presented with respect to a few machine-learning tools, the principles presented herein may be applied to other machine-learning tools.

In some example embodiments, different machine-learning tools may be used. For example, Logistic Regression (LR). Naive-Bayes, Random Forest (RF), neural networks (NN), matrix factorization, and Support Vector Machines (SVM) tools may be used for classifying or scoring job postings.

Two common types of problems in machine learning are classification problems and regression problems. Classification problems, also referred to as categorization problems, aim at classifying items into one of several category values (for example, is this object an apple or an orange). Regression algorithms aim at quantifying some items (for example, by providing a value that is a real number). The machine-learning algorithms utilize the training data 112 to find correlations among identified features 102 that affect the outcome.

The machine-learning algorithms utilize features 102 for analyzing the data to generate assessments 120. A feature 102 is an individual measurable property of a phenomenon being observed. The concept of a feature is related to that of an explanatory variable used in statistical techniques such as linear regression. Choosing informative, discriminating, and independent features is important for effective operation of the MLP in pattern recognition, classification, and regression. Features may be of different types, such as numeric features, strings, and graphs.

In one example embodiment, the features 102 may be of different types and may include one or more of words of the message 103, message concepts 104, communication history 105, past user behavior 106, subject of the message 107, other message attributes 108, sender 109, and user data 110.

The machine-learning algorithms utilize the training data 112 to find correlations among the identified features 102 that affect the outcome or assessment 120. In some example embodiments, the training data 112 includes labeled data, which is known data for one or more identified features 102 and one or more outcomes, such as detecting communication patterns, detecting the meaning of the message, generating a summary of the message, detecting action items in the message, detecting urgency in the message, detecting a relationship of the user to the sender, calculating score attributes, calculating message scores, etc.

With the training data 112 and the identified features 102, the machine-learning tool is trained at operation 114. The machine-learning tool appraises the value of the features 102 as they correlate to the training data 112. The result of the training is the trained machine-learning program 116.

When the machine-learning program 116 is used to perform an assessment, new data 118 is provided as an input to the trained machine-learning program 116, and the machine-learning program 116 generates the assessment 120 as output. For example, when a message is checked for an action item, the machine-learning program utilizes the message content and message metadata to determine if there is a request for an action in the message.

Machine learning techniques train models to accurately make predictions on data fed into the models (e.g., what was said by a user in a given utterance; whether a noun is a person, place, or thing; what the weather will be like tomorrow). During a learning phase, the models are developed against a training dataset of inputs to optimize the models to correctly predict the output for a given input. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset.

Models may be run against a training dataset for several epochs (e.g., iterations), in which the training dataset is repeatedly fed into the model to refine its results. For example, in a supervised learning phase, a model is developed to predict the output for a given set of inputs, and is evaluated over several epochs to more reliably provide the output that is specified as corresponding to the given input for the greatest number of inputs for the training dataset. In another example, for an unsupervised learning phase, a model is developed to cluster the dataset into n groups, and is evaluated over several epochs as to how consistently it places a given input into a given group and how reliably it produces the n desired clusters across each epoch.

Once an epoch is run, the models are evaluated and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several other machine learning algorithms that may be applied with the present disclosure, including linear regression, random forests, decision tree learning, neural networks, deep neural networks, etc.

Each model develops a rule or algorithm over several epochs by varying the values of one or more variables affecting the inputs to more closely map to a desired result, but as the training dataset may be varied, and is preferably very large, perfect accuracy and precision may not be achievable. A number of epochs that make up a learning phase, therefore, may be set as a given number of trials or a fixed time/computing budget, or may be terminated before that number/budget is reached when the accuracy of a given model is high enough or low enough or an accuracy plateau has been reached. For example, if the training phase is designed to run n epochs and produce a model with at least 95 accuracy, and such a model is produced before the n^{th} epoch, the learning phase may end early and use the produced model satisfying the end-goal accuracy threshold. Similarly, if a given model is inaccurate enough to satisfy a random chance threshold (e.g., the model is only 55% accurate in determining true/false outputs for given inputs), the learning phase for that model may be terminated early, although other models in the learning phase may continue training. Similarly, when a given model continues to provide similar accuracy or vacillate in its results across multiple epochs—having reached a performance plateau—the learning phase for the given model may terminate before the epoch number/computing budget is reached.

Once the learning phase is complete, the models are finalized. In some example embodiments, models that are finalized are evaluated against testing criteria. In a first example, a testing dataset that includes known outputs for its inputs is fed into the finalized models to determine an accuracy of the model in handling data that is has not been trained on. In a second example, a false positive rate or false negative rate may be used to evaluate the models after finalization. In a third example, a delineation between data clusterings is used to select a model that produces the clearest bounds for its clusters of data.

FIG. 2 illustrates an example neural network 204, in accordance with some embodiments. As shown, the artificial neural network 204 receives, as input, source domain data 202. The input is passed through a plurality of layers 206 to arrive at an output. Each layer 206 includes multiple neurons 208. The neurons 208 receive input from neurons of a previous layer and apply weights to the values received from those neurons in order to generate a neuron output, The neuron outputs from the final layer 206 are combined to generate the output of the artificial neural network 204.

As illustrated at the bottom of FIG. 2, the input is a vector x. The input is passed through multiple layers 206, where weights are applied to the input to each layer to arrive at f¹(x), f²(x), . . . , fⁱ⁻¹(x), until finally the output f(x) is computed.

In some example embodiments, the artificial neural network 204 (e.g., deep learning, deep convolutional, or recurrent neural network) comprises a series of neurons 208, such as Long Short Term Memory (LSTM) nodes, arranged into a network. A neuron 208 is an architectural element used in data processing and artificial intelligence, particularly machine learning, which includes memory that may determine when to “remember” and when to “forget” values held in that memory based on the weights of inputs provided to the given neuron 208. Each of the neurons 208 used herein are configured to accept a predefined number of inputs from other neurons 208 in the artificial neural network 204 to provide relational and sub-relational outputs for the content of the frames being analyzed. Individual neurons 208 may be chained together and/or organized into tree structures in various configurations of neural networks to provide interactions and relationship learning modeling for how each of the frames in an utterance are related to one another.

For example, an LSTM node serving as a neuron includes several gates to handle input vectors (e.g., phonemes from an utterance), a memory cell, and an output vector (e.g., contextual representation). The input gate and output gate control the information flowing into and out of the memory cell, respectively, whereas forget gates optionally remove information from the memory cell based on the inputs from linked cells earlier in the artificial neural network. Weights and bias vectors for the various gates are adjusted over the course of a training phase, and once the training phase is complete, those weights and biases are finalized for normal operation. One of skill in the art will appreciate that neurons and neural networks may be constructed programmatically (e.g., via software instructions) or via specialized hardware linking each neuron to form the artificial neural network.

Neural networks utilize features for analyzing the data to generate assessments (e.g., recognize units of speech). A feature is an individual measurable property of a phenomenon being observed. The concept of feature is related to that of an explanatory variable used in statistical techniques such as linear regression. Further, deep features represent the output of nodes in hidden layers of the deep neural network.

A neural network, sometimes referred to as an artificial neural network, is a computing system/apparatus based on consideration of biological neural networks of animal brains. Such systems/apparatus progressively improve performance, which is referred to as learning, to perform tasks, typically without task-specific programming. For example, in image recognition, an artificial neural network may be taught to identify images that contain an object by analyzing example images that have been tagged with a name for the object and, having learnt the object and name, may use the analytic results to identify the object in untagged images. An artificial neural network is based on a collection of connected units called neurons, where each connection, called a synapse, between neurons can transmit a unidirectional signal with an activating strength that varies with the strength of the connection. The receiving neuron can activate and propagate a signal to downstream neurons connected to it, typically based on whether the combined incoming signals, which are from potentially many transmitting neurons, are of sufficient strength, where strength is a parameter.

A deep neural network (DNN) is a stacked neural network, which is composed of multiple layers. The layers are composed of nodes, which are locations where computation occurs, loosely patterned on a neuron in the human brain, which fires when it encounters sufficient stimuli. A node combines input from the data with a set of coefficients, or weights, that either amplify or dampen that input, which assigns significance to inputs for the task the algorithm is trying to learn. These input-weight products are summed, and the sum is passed through what is called a node's activation function, to determine whether and to what extent that signal progresses further through the network to affect the ultimate outcome. A DNN uses a cascade of many layers of non-linear processing units for feature extraction and transformation. Each successive layer uses the output from the previous layer as input. Higher-level features are derived from lower-level features to form a hierarchical representation. The layers following the input layer may be convolution layers that produce feature maps that are filtering results of the inputs and are used by the next convolution layer.

In training of a DNN architecture, a regression, which is structured as a set of statistical processes for estimating the relationships among variables, can include a minimization of a cost function. The cost function may be implemented as a function to return a number representing how well the artificial neural network performed in mapping training examples to correct output. In training, if the cost function value is not within a pre-determined range, based on the known training images, backpropagation is used, where backpropagation is a common method of training artificial neural networks that are used with an optimization method such as a stochastic gradient descent (SGD) method.

Use of backpropagation can include propagation and weight update. When an input is presented to the artificial neural network, it is propagated forward through the artificial neural network, layer by layer, until it reaches the output layer. The output of the artificial neural network is then compared to the desired output, using the cost function, and an error value is calculated for each of the nodes in the output layer, The error values are propagated backwards, starting from the output, until each node has an associated error value which roughly represents its contribution to the original output. Backpropagation can use these error values to calculate the gradient of the cost function with respect to the weights in the artificial neural network. The calculated gradient is fed to the selected optimization method to update the weights to attempt to minimize the cost function.

FIG. 3 illustrates the training of an image recognition machine learning program, in accordance with some embodiments. The machine learning program may be implemented at one or more computing machines. Block 302 illustrates a training set, which includes multiple classes 304, Each class 304 includes multiple images 306 associated with the class. Each class 304 may correspond to a type of object in the image 306 (e.g., a digit 0-9, a man or a woman, a cat or a dog, etc.). In one example, the machine learning program is trained to recognize images of the presidents of the United States, and each class corresponds to each president (e.g., one class corresponds to Barack Obama, one class corresponds to George W. Bush, etc.). At block 308 the machine learning program is trained, for example, using a deep neural network. At block 310, the trained classifier, generated by the training of block 308, recognizes an image 312, and at block 314 the image is recognized. For example, if the image 312 is a photograph of Bill Clinton, the classifier recognizes the image as corresponding to Bill Clinton at block 314.

FIG. 3 illustrates the training of a classifier, according to some example embodiments. A machine learning algorithm is designed for recognizing faces, and a training set 302 includes data that maps a sample to a class 304 (e.g., a class includes all the images of purses). The classes may also be referred to as labels. Although embodiments presented herein are presented with reference to object recognition, the same principles may be applied to train machine-learning programs used for recognizing any type of items.

The training set 302 includes a plurality of images 306 for each class 304 (e.g., image 306), and each image is associated with one of the categories to be recognized (e.g., a class). The machine learning program is trained 308 with the training data to generate a classifier 310 operable to recognize images. In some example embodiments, the machine learning program is a DNN.

When an input image 312 is to be recognized, the classifier 310 analyzes the input image 312 to identify the class (e.g., class 314) corresponding to the input image 312.

FIG. 4 illustrates the feature-extraction process and classifier training, according to some example embodiments. Training the classifier may be divided into feature extraction layers 402 and classifier layer 414. Each image is analyzed in sequence by a plurality of layers 406-413 in the feature-extraction layers 402.

With the development of deep convolutional neural networks, the focus in face recognition has been to learn a good face feature space, in which faces of the same person are close to each other, and faces of different persons are far away from each other. For example, the verification task with the LFW (Labeled Faces in the Wild) dataset has been often used for face verification.

Many face identification tasks (e.g., associated with the datasets MegaFace and LFW) are based on a similarity comparison between the images in the gallery set and the query set, which is essentially a K-nearest-neighborhood (KNN) method to estimate the person's identity. In the ideal case, there is a good face feature extractor (inter-class distance is always larger than the intra-class distance), and the KNN method is adequate to estimate the person's identity.

Feature extraction is a process to reduce the amount of resources required to describe a large set of data. When performing analysis of complex data, one of the major problems stems from the number of variables involved. Analysis with a large number of variables generally requires a large amount of memory and computational power, and it may cause a classification algorithm to ovcrfit to training samples and generalize poorly to new samples. Feature extraction is a general term describing methods of constructing combinations of variables to get around these large data-set problems while still describing the data with sufficient accuracy for the desired purpose.

In some example embodiments, feature extraction starts from an initial set of measured data and builds derived values (features) intended to be informative and non-redundant, facilitating the subsequent learning and generalization steps. Further, feature extraction is related to dimensionality reduction, such as be reducing large vectors (sometimes with very sparse data) to smaller vectors capturing the same, or similar, amount of information.

Determining a subset of the initial features is called feature selection. The selected features are expected to contain the relevant information from the input data, so that the desired task can be performed by using this reduced representation instead of the complete initial data. DNN utilizes a stack of layers, where each layer performs a function. For example, the layer could be a convolution, a non-linear transform, the calculation of an average, etc. Eventually this DNN produces outputs by classifier 414. In FIG. 4, the data travels from left to right and the features are extracted. The goal of training the artificial neural network is to find the parameters of all the layers that make them adequate for the desired task.

As shown in FIG. 4, a “stride of 4” filter is applied at layer 406, and max pooling is applied at layers 407-413. The stride controls how the filter convolves around the input volume. “Stride of 4” refers to the filter convolving around the input volume four units at a time. Max pooling refers to down-sampling by selecting the maximum value in each max pooled region.

In some example embodiments, the structure of each layer is predefined. For example, a convolution layer may contain small convolution kernels and their respective convolution parameters, and a summation layer may calculate the sum, or the weighted sum, of two pixels of the input image. Training assists in defining the weight coefficients for the summation.

One way to improve the performance of DNNs is to identify newer structures for the feature-extraction layers, and another way is by improving the way the parameters are identified at the different layers for accomplishing a desired task. The challenge is that for a typical neural network, there may be millions of parameters to be optimized. Trying to optimize all these parameters from scratch may take hours, days, or even weeks, depending on the amount of computing resources available and the amount of data in the training set.

FIG. 5 illustrates a circuit block diagram of a computing machine 500 in accordance with some embodiments. In some embodiments, components of the computing machine 500 may store or be integrated into other components shown in the circuit block diagram of FIG. 5. For example, portions of the computing machine 500 may reside in the processor 502 and may be referred to as “processing circuitry.” Processing circuitry may include processing hardware, for example, one or more central processing units (CPUs), one or more graphics processing units (CPUs), and the like. In alternative embodiments, the computing machine 500 may operate as a standalone device or may be connected (e.g., networked) to other computers. In a networked deployment, the computing machine 500 may operate in the capacity of a server, a client, or both in server-client network environments. In an example, the computing machine 50( )may act as a peer machine in peer-to-peer (P2P) (or other distributed) network environment. In this document, the phrases P2P, device-to-device (D2D) and sidelink may be used interchangeably. The computing machine 500 may be a specialized computer, a personal computer (PC), a tablet PC, a personal digital assistant (PDA), a mobile telephone, a smart phone, a web appliance, a network router, switch or bridge, or any machine capable of executing instructions (sequential or otherwise) that specify actions to be taken by that machine.

Examples, as described herein, may include, or may operate on, logic or a number of components, modules, or mechanisms. Modules and components are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems/apparatus (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” (and “component”) is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

The computing machine 500 may include a hardware processor 502 (e.g., a central processing unit (CPU), a GPU, a hardware processor core, or any combination thereof), a main memory 504 and a static memory 506, some or all of which may communicate with each other via an interlink (e.g., bus) 508. Although not shown, the main memory 504 may contain any or all of removable storage and non-removable storage, volatile memory or non-volatile memory. The computing machine 500 may further include a video display unit 510 (or other display unit), an alphanumeric input device 512 (e.g., a keyboard), and a user interface (UI) navigation device 514 (e.g., a mouse). In an example, the display unit 510. input device 512 and navigation device 514 may be a touch screen display. The computing machine 500 may additionally include a storage device (e.g., drive unit) 516, a signal generation device 518 (e.g., a speaker), a network interface device 520, and one or more sensors 521, such as a global positioning system (GPS) sensor, compass, accelerometer, or other sensor. The computing machine 500 may include an output controller 528, such as a serial (e.g., universal serial bus (USB), or other wired or wireless (e.g., infrared (IR), near field communication (NFC), etc.) connection to communicate or control one or more peripheral devices (e.g., a printer, card reader, etc.).

The drive unit 516 (e.g., a storage device) may include a machine readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the computing machine 500. In an example, one or any combination of the hardware processor 502, the main memory 504, the static memory 506, or the storage device 516 may constitute machine readable media.

While the machine readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the computing machine 500 and that cause the computing machine 500 to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks. In some examples, machine readable media may include non-transitory machine readable media. In some examples, machine readable media may include machine readable media that is not a transitory propagating signal.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 520 may include one or more physical jacks (e.g., Ethernet, coaxial, or phone jacks) or one or more antennas to connect to the communications network 526.

Sleep plays a vital role in human health, both mental and physical. Sleep disorders like sleep apnea are increasing in prevalence, with the rapid increase in factors like obesity. Sleep apnea is most commonly treated with Continuous Positive Air Pressure (CPAP) therapy. Presently, however, there is no mechanism to monitor a patient's progress with CPAP. Accurate detection of sleep stages from CPAP flow signal is useful for such a mechanism. Some aspects propose an automated sleep staging model based on the flow signal.

Deep neural networks have recently shown high accuracy on sleep staging by eliminating handcrafted features. However, these methods focus exclusively on extracting informative features from the input signal, without paying much attention to the dynamics of sleep stages in the output sequence. Some aspects propose an end-to-end framework that uses a combination of deep convolution and recurrent neural networks to extract high-level features from raw flow signal with a structured output layer based on a conditional random field to model the temporal transition structure of the sleep stages. Some aspects use a model that can be augmented to the previous sleep staging deep learning methods. Some aspects accurately track sleep metrics like sleep efficiency calculated from sleep stages that can be deployed for monitoring the response of CPAP therapy on sleep apnea patients.

Sleep plays a fundamental role in the physical and emotional recovery of the human body. Sleep deprivation or poor quality of sleep adversely affect the quality of life. Outside of the wake state, sleep can be divided into four stages: Rapid Eye Movement (REM), and Nan-REM (NREM) stages 1, 2, and 3. Due to transitory nature of NREM stage 1, stages 1 and 2 are often grouped and classified as light sleep, as compared to deep sleep for NREM stage 3, Each stage has its role in the recovery process, e.g., REM sleep helps in memory consolidation and emotion regulation while deep sleep helps with physical recovery processes. Understanding of a subject's sleep states and their dynamics is necessary for identifying and monitoring various sleep-related disorders such as sleep apnea. The economic cost of sleep-related disorders is enormous. One of the leading cost burdens is due to Obstructive Sleep Apnea (OSA). OSA is a disorder in which an airway collapses during inhalation resulting in a reduced oxygen supply to the brain, forcing the patient to wake, causing interrupted sleep. OSA poses a severe risk. For example, OSA is associated with higher rates of heart attacks. Despite the severity of the condition, it is a mostly undiagnosed disease with an estimated 5%-20% prevalence rates among the population with an estimated cost burden of $150 billion per year in the United States alone.

FIG. 6 illustrates an example use case 600 of a Continuous Positive Air Pressure (CPAP) device 610, in accordance with some embodiments. FIG. 6 illustrates an application use case of the model according to some aspects. A patient 602 undergoes Polysomnography (PSG) 604 to ascertain the sleep disorders and is diagnosed with Sleep Apnea 608. Healthcare provider 606 recommends the CPAP therapy that involves the CPAP device 610. A flow signal 614 can be obtained from the device daily (or at any other frequency) for monitoring purposes. By adding the automated sleep staging 612, some aspects can provide the healthcare provider 606 with a means for continuous monitoring of the patient 602 (with the patient's affirmative consent).

One prevalent and effective treatment for OSA is Continuous Positive Airway Pressure (CPAP) therapy. In a CPAP therapy, a user wears a mask, connected to a flow generating device, which delivers an adaptive pressure to prevent the collapse of the airway and track signals like daily airflow pressure (flow signal) data. This data contains valuable information transmitted to health-care professionals for monitoring the subject's respiratory patterns. However, in some schemes, it is not being utilized actively to monitor the efficacy of patient therapy or sleep quality which in-turn could pave way for an intervention as done in other health-care areas. The key to measure the effectiveness of CPAP therapy is to assess the sleep quality by determining the sleep stages. Some aspects are directed to determining sleep stages from CPAP-available signals. Determination of sleep stages has been typically performed on data obtained from Polysomnograms (PSG), which involves an overnight measurement of a variety of biological signals during sleep. The gold standard for securing sleep stages is for trained sleep experts to manually annotate PSG data. However, this may be very expensive.

Prior studies on sleep staging have focused on automating the annotations by using reduced number of sensors from PSG including Electroencephalography (EEG) or using her more comfortable devices like actigraphy, cardio-respiratory sensors, or no-contact sensors. However, all of these approaches do not have a direct use case—they require additional devices to provide data for sleep staging. Some embodiments use the CPAP-available flow signal to identify sleep stages automatically. CPAP users can know about their sleep health by learning about their sleep states, while the health-care providers can track longitudinal sleep health and overall success of CPAP therapy. FIG. 6 shows a schematic of the application of some embodiments.

On the technical front, some schemes use artificial neural networks with hand-crafted features. In some cases, deep neural networks have been used for end-to-end learning without manual feature engineering mainly based on convolutional neural networks (CNN). Hybrid recurrent-convolutional neural networks (R-CNN) methods that use CNN as base network fed to the recurrent networks have shown human-expert level accuracy on PSG. Adversarial training with R-CNN proposed may deliver results on RF-signals. These schemes focus on learning informative abstract features from the input signal making predictions at each time step independent of the previous sleep state. However, sleep states have a. strong transition structure. Some aspects take into account the dynamics of the sleep states, giving the deep learning methods an essential source of information.

Some aspects are directed to a new neural network architecture based on chain-structured conditional random field (CRF) that explicitly models the temporal dynamics in the sleep states, over the deep convolutional neural network to learn high-level abstract features from CPAP flow signals and a recurrent neural network to encode temporal context in these features. The entire Neural CRF (CNN-RNN-CRF) network is trained for sleep staging in an end-to-end fashion.

The disclosed Neural CRF method shows a substantial improvement over the state-of-art when applied to the CPAP flow signal for sleep staging. Further, some aspects improve the performance using a class distribution cost-sensitive prior to deal with the imbalanced distribution of sleep stages and using a domain dependent regularization over the CRF parameters. In summary, some aspects make the following contributions: (a) While some schemes have entirely focused on extracting best features from the input signals, the disclosed technique, in some aspects, demonstrate that jointly modeling the dynamics of the output sleep stages can substantially increase the performance. Some aspects can be added to other deep learning models that are competing in the input space. (b) Some aspects use a CNN architecture along with a recurrent layer to extract high-level features from CPAP flow signals.

Some aspects include the first study on automatic sleep staging using the respiratory flow signal. This model has a direct existing application use case—providing healthcare professionals a way to track the patients undergoing sleep apnea treatment through CPAP devices. By directly linking CPAP flow data to sleep stages, this technique has the potential to illustrate an improvement in sleep and create an interest in investigating the cognitive and neuronal benefits of adhering to CPAP therapy.

FIG. 7 illustrates an example sleep state transition diagram 700, in accordance with some embodiments. FIG. 7 is a transition diagram for OSA patients with non-REM states 1 and 2 combined as Light (L) sleep state. The four sleep states shown are: (W)ake, (R)EM, (L)ight and (D)eep. 100741 PSG is the gold standard for assessment of sleep quality and diagnosis of specific sleep disorders. It requests the subject to spend a night in a sleep lab with a variety of sensors attached to collect data about the biological processes during sleep. In clinical practice, several levels of health-care sleep professionals visually annotate the data in 30-second epochs to ascertain the sleep stages. Sleep staging is a labor-intensive process with limitations due to inter-expert variability. The PSG process has a very high overhead in terms of cost and convenience. Some schemes focus on reducing the number of sensors to asses sleep stages as an alternative to PSG.

Some aspects use a new source signal, namely nasal airflow (flow) This flow signal is a measure of respiratory effort, similar to chest-band based sensors that measure the respiratory patterns of subjects during sleep. Unlike other methods, however, the flow signal has an existing use-case; persons with OSA who are regularly using CPAP therapy. One approach can provide a mechanism for continuously monitoring these patients' sleep health and response to the therapy with significantly improved accuracy for sleep staging and very high accuracy for sleep efficiency.

In some schemes, deep learning methods have focused on extracting the best possible features from the input signal ignoring or paying limited attention to the context of each segment and dynamics of the sleep states. However, sleep stage transitions have a strong dependency structure with many transitions having extremely low probability. For example, the transition between REM and deep sleep may implement an intermediate step; having contiguous REM and deep sleep epochs would require the unlikely occurrence of several transitions taking place inside the same epoch. Furthermore, some detectable events like rapid eye movements, arousals or K-spindles dictate epoch-to-epoch stability or stage transitions. In such scenarios, it is essential to take into account both the input signal and the dynamics of sleep states. R-CNN models assume that recurrent connections can capture the sleep state transition structure while attempts with convolution networks assume that taking the immediate neighboring segments into account should suffice.

Some aspects use a conditional random field model that does joint modeling of the sleep stages for the entire duration of sleep, trained end-to-end with a deep R-CNN network. Some aspects substantially increase the model's performance over adversarial or baseline R-CNN. This approach can he augmented to any of the existing deep learning methods.

Some aspects combine a convolutional neural network (CNN), a recurrent neural network (RNN), and a conditional random field (CRF) in a single architecture that is trained end-to-end.

Let the input flow signal time-series be x=(x₁, x₂, . . . , xₙ), where xᵢ's are signal values sampled at 32 Hertz, i.e., 32 signal values per second. Annotation of sleep stages is done for each 30-second epoch corresponding to 30* 32=960 signal values in x. An example flow signal and corresponding sleep stages for a night's sleep is shown in FIG. 9. The computational task is to annotate the signal time-series for each 30-second epoch with a label yi as one of the four sleep stages: Wake (W), REM (R), Light Sleep (L), and Deep Sleep (D). In other words, some aspects label the sequence x=(x₁, x₂, . . . , xₙ) with y=(y₁, y₂, . . . , yₘ), where in m=n/960. In some cases, in m=900 and n=900×960=864,000. Some aspects denote the set of sleep states as K={W, R, D} throughout this document.

Convolution neural networks have been used extensively for a variety of tasks in computer vision, natural language processing, and time-series analysis. Some aspects use convolution layers to extract high-level abstract features that are then fed to the recurrent layer. The convolutional neural network takes the flow signal time-series x=(x₁, x₂, . . . , xₙ) as input and passes it through a sequence of convolution layers to generate in abstract feature vectors Z=(z₁, z₂, . . . , zₘ) that are fed to a recurrent neural network for further processing.

Some aspects use an architecture as the base convolution neural network. FIG. 8 illustrates an example artificial neural network 800 that may be used in sleep stage modeling. Each convolution layer 804, 806, 808, 810, and 812 involves a one-dimensional (1D) convolution operation followed by a rectified linear unit (ReLU) non-linear activation, a dropout, and (optionally) a max-pooling operation. Let RT be the input sequence of length m to a convolution layer (at any depth) with j-th kernel K^{j} of size W and stride size s. The 1D convolution operation at t ∈ {1, 2, . . . , T} is defined as shown in Equation 1.

$${Φ}_{i} = f ( {\sum }_{i = 1}^{W} {K}_{i}^{j} {𝒳}_{t + i \cdot s - 1} + b ) \qquad ( 1 )$$

FIG. 8 illustrates one deep learning model architecture leveraging an artificial neural network 800, with an input 802 provided to CNN layers 804, 806, 808, 810, and 812, GRU connections 814, and CRF 816 for end-to-end learning. The CRF 816 outputs the sleep stages 818. The CNN layers 804, 806, 808, 810, and 812 leverage max-pooling 820. W refers to the Kernel size, S refers to the stride size, and C refers to the channel size at each convolution layer. The numbers indicated at the end of each convolution layer represent the size of output at each layer.

In Equation 1, b is the kernel K's bias and f ( )represents the activation function ReLU defined as f(z)=max(z, 0). The outputs of convolution at each t are concatenated to produce a feature map for each of the N kernels.

Convolution operations help extract the local features of time-series signal in a location invariant way. Strides s and filter width W capture the transitions in the input and receptive field or catchment of the convolution operation, respectively.

Some aspects utilize dropout on the rectified activations to avoid overfitting. For some layers, the convolution-pooling operations are succeeded by a max-pooling operation.

Additionally, some aspects use residual connections between two layers so that a new layer added to the network learns something new. They also help with the diminishing gradient of preceding layers problem in deep convolution networks by forcing the network to learn the identity mapping. Formally, let be the input to a convolution layer, and F(x) represent the output of repeated convolution-pooling layers. The residual connection is defined as shown in Equation 2.

X″= [symbol] (X)+U^{T}X (2)

In Equation 2, U is a transformation matrix that is used to bring X to the same dimensions as of F(X). In case both have the same dimensions, U becomes an identity matrix. Residual connections are usually used after one or two convolution-pooling layers. Some aspects use it between the second layer 806 and the fourth layer 810 of the artificial neural network 800 of FIG. 8.

As shown in FIG. 8, the first convolution layer 804 in the artificial neural network 800 uses 256 different kernels (i.e., output channels) each of size 10 and stride 2. This generates 432,000 feature values in each feature map. The second convolution layer then applies a kernel of window size 10 and stride 2 to each feature map and reduces the number of features in each feature map to 43,200. This process continues until the last convolution layer, which generates m=900 features in each feature map. In other words, the output of the CNN is Z ∈ R^{256*900}.

Recurrent neural networks are used to model inputs with sequential nature. Since the data is a time-series sequence, some aspects use the recurrent layer to model the temporal nature of the signal that builds on the high-level features from the convolution layers. The recurrent layer takes the feature vectors Z=(z₁, z₂, . . . , zₙ) produced by the preceding ResNet CNN as input, and computes a representation. hₜ at every time step t by combining the current input zₜ with the output of the previous time step hₜ₋₁. The recurrent units model the temporal dynamics of the input signal, h working on the sharp feature maps of the CNN.

Some aspects use Gated Recurrent Units (GRUs) as the recurrent units. A GRU may have two gates: an update gate (u) and a reset gate (r) apart from the hidden cell state h. It combines the forget gate and the input gate of the popular Long Short-Term Memory (LSTM) unit into one update gate. The update equations for a GRU may be written as:

uₜ=σ(W_{z}zₜ+U_{z}hₜ₋₁+b_{z}) (3)

rₜ=σ(Wᵣzₜ+Uᵣhₜ₋₁+bᵣ) (4)

[symbol]

=2σ(Wₕzₜ+rₜ ⊙ Uₕhₜ₋₁+bₕ) (5)

hₜ=uₜ ⊙ hₜ₋₁+(1-uₜ) ⊙ hₜ (6)

In Equations 3-6, σ( )is the sigmoid activation function defined as σ(x)=1/(1+e^{-x}), W's and U's are weight matrices, b's are biases, and denotes the Hadamard or element-wise product. GRUs have been shown to be much faster owing to reduced number of parameters and perform at par with LSTMs.

The vector hₜ effectively represents each 30-second epoch in context, which can be used to classify the epoch into one of the sleep stages using a softmax layer. Formally, the probability of k-th class for classifying into K sleep stages is

$$p ( {y}_{t} = k \vert {h}_{t} , {W}_{o} ) = \frac{exp ( {W}_{o , k} {h}_{t} + {b}_{k} )}{{\sum }_{k = 1}^{K} exp ( {W}_{o , k} {h}_{t} + {b}_{k} )} \qquad ( 7 )$$

In Equation 7, W are the classifier weights, and b are the bias terms. Some aspects minimize the negative log likelihood (NLL) of the gold labels. The NLL for one data point (x, y) is:

$${ℒ}_{c} ( θ ) = - {\sum }_{k = 1}^{K} {\sum }_{t = 1}^{m} ℐ ( {y}_{t} = k ) log p ( {y}_{t} = k \vert x , θ ) \qquad ( 8 )$$

In Equation 8, 0 denotes the set of model parameters, and (y=k) is an indicator function to encode the gold labels: (y=k)=1 if the gold label y=k, otherwise 0. The loss function minimizes the cross-entropy between the predicted distribution and the target distribution (I.e., gold labels). This combined architecture (i.e., an RNN layer on top of a CNN) is referred to as R-CNN.

The R-CNN model presented above works in the input signal space and predicts the sleep stage for each time step independently based on the corresponding RNN hidden state. Although it considers the input context through recurrent layers, it is oblivious to the dynamics in the output space, i.e., dynamics of the sleep stages.

Some schemes using deep neural networks have focused entirely on extracting the best features from the input signals like EOG, ECG, or RF signals for predicting the sleep stage. Like R-CNN, these methods make independent (as opposed to collective) decisions. In some cases, this approach is not optimal especially when there are strong dependencies across output labels. It is known that the sleep stage transitions have a strong dependency structure. For example, a number of transitions are not allowed as can be seen in FIG. 7. Also, there could be complex dependencies like the long-term cyclical effect of events like arousal or K-complex spindles on deep and REM sleep states. Exploiting this transition structure for an accurate sleep staging is important. Also, because of local normalization (i.e., softmax in Equation 7) these models might suffer from the so-called label bias problem.

Instead of modeling classification decisions independently, some aspects model them jointly using a conditional random field (CRF). In the disclosed neural network, some aspects put the CRF layer above the recurrent layer of R-CNN, and train the whole network end-to-end. CRFs have been shown to be able to utilize the global temporal context for maximizing sequence probabilities, relying upon first- or higher-order Markov assumptions over the output label transitions.

The input to the neural CRF layer is a sequence of hidden states H=(h₁, h₂, , hₘ) from the GRU-based recurrent layer, and the corresponding label sequence is y=(y₁, y₂, , yₘ). The compatibility of the input feature H and an output label yₜ from among W, R, D, L at time step t is computed by the unary (node) potential defined as shown in Equation 9, where φ(·)denotes the feature vector computed from the input and the sleep stage labels, and wₙ is the associated weight vector. Here, Ψₙ(yₜ H, wₙ, bₙ) can be considered as a score (unnormalized probability) given to label yₜ. Applying the node potential to all nodes in the sequence generates a matrix S of size m×|K|, where K is the set of sleep stages/classes (in one case |K|=4), and Si,j corresponds to the score of the j-th class for input hi.

Ψₙ(yₜ|H, wₙ, bₙ)=exp(wₙ^{T}ϕ(yₜ, H)+bₙ) (9)

To model dynamics in the label sequence, some aspects define edge potentials between and yₜ₋₁ as shown in Equation 10, where φ(yₜ₋₁, yₜ, H) denotes edge features with wₙ being the corresponding weight vector. The edge potential computes a score for each possible edge transition in a matrix of size |K|*|K|. The joint conditional probability for the sequence is defined as shown in Equation 11, where Z(H, wₙ, θ) is the global normalization constant (partition function) derived as the sum over all possible sequences and θ denotes the set of all parameters in the complete (R-CNN-CRF) network.

$$\begin{matrix} {Ψ}_{e} ( {y}_{t - 1} , {y}_{t} \vert H , {w}_{e} , {b}_{e} ) = exp ( {w}_{e}^{T} φ ( {y}_{t - 1} , {y}_{t} , H ) + {b}_{e} ) & ( 10 ) \\ p ( y \vert H , θ ) = \frac{1}{Z ( H , θ )} {\prod }_{t = 1}^{m} {Ψ}_{n} ( {y}_{t} \vert H , {w}_{n} , {b}_{n} ) {\prod }_{t = 2}^{m} {Ψ}_{e} ( {y}_{t - 1} , {y}_{t} \vert H , {w}_{e} , {b}_{e} ) & ( 11 ) \\ Z ( H , θ ) = {\sum }_{y} {\prod }_{t = 1}^{m} {Ψ}_{n} ( {y}_{t} \vert H , {w}_{n} , {b}_{n} ) {\prod }_{t = 2}^{m} {Ψ}_{e} ( {y}_{t - 1} , {y}_{t} \vert H , {w}_{e} , {b}_{e} ) & ( 12 )\end{matrix}$$

This global normalization constrains the distribution to a valid probability distribution and helps overcome the label bias problem of locally normalized models. The negative log-likelihood for one data point can be expressed as shown in Equations 13-14.

$$\begin{matrix}ℒ ( θ ) = - log p ( y \vert H , θ ) = log Z - {\sum }_{t = 1}^{m} {w}_{n}^{T} φ ( {y}_{t} , H ) - {b}_{n} & ( 13 ) \\ - {\sum }_{t = 2}^{m} {w}_{e}^{T} φ ( {y}_{t - 1} , {y}_{t} , H ) - {b}_{e} & ( 14 )\end{matrix}$$

Note that the objective in Equation 14 is convex with respect to the CRF parameters θ^{j}=wₑ, wₙ, bₑ, bₙ assuming the inputs from the R-CNN (i.e., Z) are fixed. In training, wₑ inputs from the R-CNN (i.e., Z) are fixed. In training, some aspects add a l₁ regularization on the CRF parameters θ^{j} to promote sparsity. The final objective can thus be written as shown in Equation 15.

$${min}_{θ} ℒ ( θ ) + λ {‖ {θ}^{′} ‖}_{1} \qquad ( 15 )$$

As can be seen in FIG. 7, a number of transitions are not observed while some have a large value making l₁ norm more appropriate to this problem compared to a l₂ norm.

The complete R-CNN-CRF network is trained end-to-end on the loss in Equation 15 by back-propagating the errors (gradients) to the R-CNN. Similar end-to-end training has shown impressive results in computer vision. Once the parameters of the network are learned, decoding the most probable sequence is performed effectively using the Viterbi algorithm.

In some cases, the class distribution of different sleep stages is skewed with REM and Deep sleep forming less than 10% of the annotations. To tackle this issue, some aspects add a class prior αₖ over the log likelihood. The class prior αₖ for k selected from 1, 2, . . . , K is estimated from the training data by Equation 16, where n_{μ} is the average number of labels in each class i.e., n/K with n being the total number of sleep labels in the training set. Some aspects incorporate the class prior in the loss from Equation 15 as shown in Equation 17. In Equation 17, [symbol] (·) is the Boolean indicator function. The priors a are inversely proportional to the number of samples of the class giving more weight-age to the under-represented classes leading to balanced learning during the training phase.

$$\begin{matrix}{α}_{k} = \frac{{n}_{μ}}{{n}_{k}} & ( 16 ) \\ {min}_{θ} - {\sum }_{k = 1}^{K} {\sum }_{t = 1}^{m} ℐ ( {y}_{t} = k ) {α}_{k} log p ( {y}_{t} = k \vert θ ) + λ {‖ {θ}^{′} ‖}_{1} & ( 17 )\end{matrix}$$

FIG. 9 illustrates example graphs 900 an example of flow signal and corresponding sleep stage annotations for a person, in accordance with some embodiments. Graph 902 shows the flow signal versus time, and graph 904 shows the sleep stage versus time.

Some of the methods described herein can be categorized into three types: (i) linear (non-deep) CRF, (ii) deep neural models with the softmax output layer, and (iii) the deep neural models with CRF output layer.

Linear (non-deep) CRF: As can be observed, the baseline CRF may perform poorly with a low accuracy of 52.4%, K of only 0.27, and higher MAE of on sleep efficiency. it can be attributed to the low representational power of the input features compared with the task-specific feature extraction of deep learning architectures.

Deep neural models with softmax output: The deep neural models improve the performance considerably over the non-deep CRF model by taking the accuracy to 71%, K to 0.49, and MAE down to 12.5. The baseline R-CNN with ResNet-GRU architecture performs the best overall. The conditional adversarial network performs at par with the R-CNN, while it poses additional training challenges because of the instability caused by adversarial training. Using the local attentional mechanism leads to a slight drop in performance, possibly due to the extra parameters. The residual connection was quite beneficial for the R-CNN model; it increased the x scores from 0.29 to 0.49.

Neural CRF models: Adding the CRF layer to the base R-CNN improves the performance substantially taking the κ score to 0.54, a 10.2% increase in relative terms. Adding second order edges in the CRF marginally improves the performance, though it increases the run-time of the model substantially. Using a cost-sensitive version of the Neural CRF increases the performance considerably by 2% in K over the Neural CRF, while the regularized cost sensitive CRF improves the performance by 4% over the Neural CRF model. Using CRF that infers the global temporal context improves the performance substantially. Adding domain dependent prior knowledge like cost-sensitive prior and sparse regularization helps bring additional gains in the model performance.

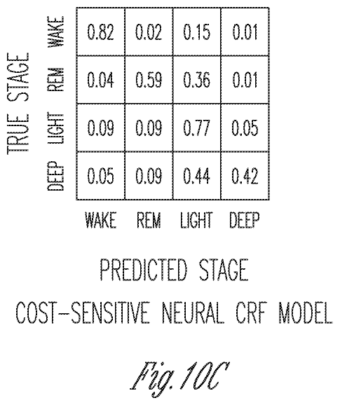

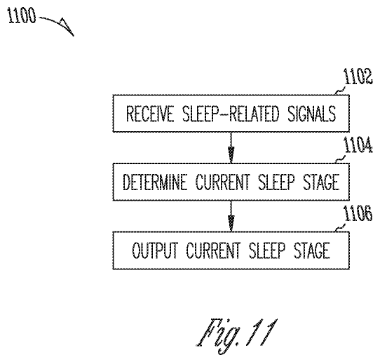

FIG. 10A illustrates an example next stage/previous stage table for a conditional random field (CRF) sleep stage transition matrix, in accordance with some embodiments.

FIG. 109 illustrates an example predicted stage/true stage table for a neural CRF model, in accordance with some embodiments.

FIG. 10C illustrates an example predicted stage/true stage table for a cost-sensitive neural CRF model, in accordance with some embodiments.

A transition matrix of the regularized Neural CRF model is shown in FIG. 10A. As can be seen, the values in the matrix assign zero scores to the non-existent transitions in FIG. 7.

The deep non-linear layers help the model to extract the feature space that is very relevant to the task as reflected by the difference in performance of the non-deep CRF vs. R-CNN. Combining the two into the Neural CRF does is very helpful in leveraging the strengths of both approaches deep learning for meaningful feature extraction in the input side, and the modeling strength of CRFs, which use global inference to model consistency in the output structure.

The precisely similar trend as above is observed with sleep efficiency MAE. The models can predict sleep efficiency metric with a reasonable accuracy—within 10%-15% of the sleep efficiency value. This is expected since the model is able to differentiate the wake state with very high accuracy, as shown by the confusion matrices in FIGS. 10B and 10C. The model can provide an accurate estimation of sleep efficiency to help health-care professionals track the response of CPAP therapy.

FIG. 10A illustrates a transition matrix from CRF of Regularized Cost-Sensitive Neural CRF from training. FIG. 10B illustrates Confusion matrices of prediction from the baseline Neural CRF model on a test dataset. FIG. 10C illustrates Confusion matrices of prediction from the cost-sensitive Neural CRF model on a test dataset.

Since the data may, in some cases, have under-represented REM and deep sleep annotations, a cost-sensitive prior may be used. The effect of this prior is demonstrated by showing the confusion matrices for the Neural CRF and the cost-sensitive Neural CRF models in FIGS. 10B-10C. By adding cost sensitive prior, the class accuracies increase across the board, significantly for the under-represented classes of REM and deep sleep. Hence, using a cost-sensitive prior for lifting the weights of the under-represented classes during training is helpful for reducing the effect of imbalanced class distributions.

The flow signal-based models are able to detect the wake state accurately and light sleep with good accuracy, but might, in some cases, have difficulty detecting the REM and deep sleep.

Other signals such as no-contact and chest band-based signals have relatively lower performance on the wake and deep sleep states detection while are able to detect the REM and light sleep states accurately. Using flow signal for the sleep staging might do better than actigraphy, since the machine is able to detect the light sleep quite accurately compared to the actigraphy, in addition to the wake sleep actigraphy has shown to detect accurately. Previous attempts on sleep apnea patients have observed lower accuracy compared to the healthy subjects since the sleep dynamics exhibited by sleep apnea patients are harder to predict than those of healthy subjects. Also, the study has a direct use case for the sleep apnea patients on CPAP treatment. Given the results on sleep efficiency task, flow signal can be used for monitoring the sleep efficiency of the patients, which is a very useful metric for the success of the therapy.

One criticism of deep learning methods comes from the black-box nature of the models. Some aspects disclose the flow signal saliency as an exercise to interpret the model's basis for prediction. In one case, the CRF layer's transition matrix (FIG. 10A) helped understand the output sleep stage dynamics learned by the model. In order to get an understanding of how the model is predicting the sleep stages from the input flow signal, some aspects use a saliency map approach to interpret CNNs for image classification. The idea is to take the gradient of the classification scores with respect to the input image to learn weights of pixels the model is “looking” at while making predictions.

Some aspects use the same approach in this setting by learning the weights of model saliency over flow signal time-series by taking gradients of the classification scores with respect to the input flow signal.

Through a visual inspection, it can be observed that the models seem to focus on two phases of the respiratory cycle, namely plateaus in flow closest to zero between inhalation and exhalation and periods of maximal change in flow rates. Respiratory rate variability and respiratory effort amplitude differ depending on stage of sleep. Thus, it is conceivable that the models may be extracting information that approximates respiratory physiology features in trying to classify sleep stage. On the other hand, the model's saliency map may also represent new and unknown phenomena that could be useful for medical researchers to investigate.

Some aspects are directed to using flow signal for automated sleep staging. Some aspects utilize a neural CRF architecture that combines the representational power of deep neural networks with the modeling strength of structured output models to get the best of both worlds. For the neural model, some aspects employ a deep CNN to learn high-level informative features from flow signals. A GRU-based RNN is used to encode features for classification by modeling temporal contextual information. The CRF jointly models the output sequence to capture temporal dynamics in the sleep states. Domain-dependent priors may be used to regularize the network.

The disclosed technique improves the classification performance over the baseline deep learning methods. Some aspects further demonstrate that using cost-sensitive prior for tackling class imbalance and sparse regularization on weights further improves the model performance. The neural model (R-CNN) and the CRF approach can be augmented to the existing methods for improved sleep staging. In terms of implications for the sleep care, the disclosed technique has an existing and immediate use case as it can be employed to track the response of patients to the CPAP therapy by automatically and accurately tracking sleep stages and overall sleep quality. Hence, the study is helpful in advancing clinical sleep research and motivates researchers to investigate the effect of CPAP on sleep architecture of subjects.

FIG. 11 is a flow chart of an example method 1100 for identifying sleep stages, in accordance with some embodiments.

At operation 1102, a computing machine (e.g., computing machine 500) receives, from a sensor (e.g., CPAP device 610) observing a person (e.g., patient 602), a plurality of sleep-related signals (e.g., flow signal 614 or any other signals detected by the CPAP device 610). The sleep-related signals may include sensor data from the sensor. The sensor data may represent air flow or air pressure. In some examples, the sensor resides on a face mask of a person who may be sleeping, and measures airflow or air pressure over the person's nose and/or mouth. The airflow or air pressure includes an oral or a nasal air pressure of the person who may be sleeping. The face mask may be a CPAP in-home therapy mask with adaptive pressure during sleep.

At operation 1104, the computing machine determines, using an artificial neural network (e.g., artificial neural network 800) and based on at least the plurality of sleep-related signals, a current sleep stage of the person. The current sleep stage is one of: wake, rapid eye movement (REM), light sleep, and deep sleep. The artificial neural network comprises a convolutional neural network (CNN), a recurrent neural network (RNN), and a conditional random field (CRF). The current sleep stage may be determined based, at least in part, on a past sleep stage of the person.

At operation 1106, the computing machine provides an output representing the current sleep stage. For example, the current sleep stage may be transmitted to a computing device of the healthcare provider 606.

In some examples, the computing machine transmits, using a wired or wireless communication interface, a control signal (e.g., corresponding to the output generated in operation 1106) based on a current sleep stage of the person. The control signal controls a device proximate to the person or the sensor (e.g., within a radius of 20 meters or 100 meters from the person or the sensor or within the same room, home or building as the person or the sensor). In some cases, the device is different from the computing machine and external to the computing machine. For example, the control signal may control one or more of: an oxygen provision device of the person, a facial pressure device of the person (e.g., on the CPAP in-home therapy mask), a lighting device in a room of the person (e.g., the person may desire different lighting in different sleep stages), a heating, ventilation, and air conditioning (HVAC) device in a room of the person (e.g., the person may desire different temperatures at different sleep stages), a white noise device in a room of the person (e.g., the person may desire different amounts of white noise at different sleep stages), and an alarm clock for waking the person (e.g., the person may desire to awaken during a time when he/she is in a specified sleep stage). The device proximate to the person may be controlled, using the control signal(s), continuously during the sleep of the person based on the current sleep stage and other factors (e.g., the current time in the night). For example, the person might wish to be awakened five minutes after the first time he/she enter light sleep between 6:00 am and 7:30 am, and may set control signal(s) transmitted to his/her alarm clock accordingly. In another example, a person might desire white noise starting when he/she first goes to sleep in the evening and ending when he/she enters REM sleep, and may set control signal(s) transmitted to a white noise machine accordingly.

In some examples, the RNN comprises a plurality of gated recurring units (GRUs). At least one GRU from the plurality of GRUs comprises an update gate and a hidden cell state. That GRU may compute a set of update equations based on an input received at the update gate and the hidden cell state.

In some examples, the artificial neural network includes the CNN followed by the RNN followed by the CRF. The CRF generates the output representing the current sleep stage. In some examples, the CNN includes a plurality of blocks, Each block includes a one-dimensional (1D) convolution, followed by a rectified linear unit (ReLU), followed by a dropout. In some cases, in at least one block from the plurality of blocks, the dropout is followed by a max-pooling.

Some embodiments are described as numbered examples (Example 1, 2, 3, etc.). These are provided as examples only and do not limit the technology disclosed herein.

Example 1 is a method. comprising: receiving sensor data representing airflow or air pressure; determining, using an artificial neural network, a current sleep stage corresponding to the sensor data, wherein the current sleep stage is one of: wake, rapid eye movement (REM), light sleep, and deep sleep, wherein the artificial neural network comprises a convolutional neural network (CNN), a recurrent neural network (RNN), and a conditional random field (CRF); and providing an output representing the current sleep stage.

In Example 2, the subject matter of Example 1 includes, wherein the sensor data is received from a sensor residing on a face mask of a person, wherein the airflow or the air pressure comprises an oral or a nasal airflow or air pressure of the person.

In Example 3, the subject matter of Examples 1-2 includes, wherein the RNN comprises a plurality of gated recurrent units (GRUs).

In Example 4, the subject matter of Example 3 includes, wherein at least one GRU from the plurality of GRUs comprises an update gate and a hidden cell state, wherein the at least one GRU computes a set of update equations based on an input received at the update gate and the hidden cell state.

In Example 5, the subject matter of Examples 1-4 includes, wherein the artificial neural network comprises the CNN followed by the RNN followed by the CRF, wherein the CRF generates the output representing the current sleep stage.

In Example 6, the subject matter of Examples 1-5 includes, wherein the CNN comprise a plurality of blocks, each block comprising a one-dimensional convolution, followed by a rectified linear unit (ReLU), followed by a dropout.

In Example 7, the subject matter of Example 6 includes, wherein, in at least one block from the plurality of blocks, the dropout is followed by a max-pooling.

In Example 8, the subject matter of Examples 1-7 includes, wherein the current sleep stage is determined based, at least in part, on a past sleep stage.

In Example 9, the subject matter of Examples 1-8 includes, transmitting, using a wired or wireless communication interface, a control signal based on the current sleep stage, the control signal to control a device proximate to a sensor from which the sensor data is received.

In Example 10, the subject matter of Example 9 includes, wherein the control signal is to control one or more of: an oxygen provision device, a facial pressure device, a lighting device, a heating, ventilation, and air conditioning (HVAC) device, a white noise device, and an alarm clock.

Example 11 is a non-transitory machine-readable medium storing instructions which, when executed by processing circuitry of one or more machines, cause the processing circuitry to perform operations comprising: receiving sensor data representing airflow or air pressure; determining, using an artificial neural network, a current sleep stage corresponding to the sensor data, wherein the current sleep stage is one of: wake, rapid eye movement (REM), light sleep, and deep sleep, wherein the artificial neural network comprises a convolutional neural network (CNN), a recurrent neural network (RNN), and a conditional random field (CRF); and providing an output representing the current sleep stage.

In Example 12, the subject matter of Example 11 includes, wherein the sensor data is received from a sensor residing on a face mask of a person, wherein the airflow or the air pressure comprises an oral or a nasal airflow or air pressure of the person,

In Example 13, the subject matter of Examples 11-12 includes, wherein the RNN comprises a plurality of gated recurrent units (GRIN).

In Example 14, the subject matter of Example 13 includes, wherein at least one GRU from the plurality of GRUs comprises an update gate and a hidden cell state, wherein the at least one GRU computes a set of update equations based on an input received at the update gate and the hidden cell state.

In Example 15, the subject matter of Examples 11-14 includes, wherein the artificial neural network comprises the CNN followed by the RNN followed by the CRF, wherein the CRF generates the output representing the current sleep stage.

In Example 16, the subject matter of Examples 11-15 includes, wherein the CNN comprise a plurality of blocks, each block comprising a one-dimensional convolution, followed by a rectified linear unit (ReLU), followed by a dropout.

Example 17 is an apparatus comprising: a data receiver to receive sensor data representing airflow or air pressure; a memory storing an artificial neural network, the artificial neural network to determine a current sleep stage corresponding to the sensor data, wherein the current sleep stage is one of: wake, rapid eye movement (REM), light sleep, and deep sleep, wherein the artificial neural network comprises a convolutional neural network (CNN), a recurrent neural network (RNN), and a conditional random field (CRF); processing circuitry to execute the artificial neural network; and an output device to provide an output representing the current sleep stage.

In Example 18, the subject matter of Example 17 includes, wherein the data receiver comprises a wireless radio or a wired connection.

In Example 19, the subject matter of Examples 17-18 includes, wherein the output device comprises a network interface card or a display port,

In Example 20, the subject matter of Examples 17-19 includes, wherein the output device is further to transmit a control signal based on the current sleep stage, the control signal to control a device external to the apparatus.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Although an embodiment has been described with reference to specific example embodiments, it will be evident that various modifications and changes may be made to these embodiments without departing from the broader spirit and scope of the present disclosure. Accordingly, the specification and drawings are to be regarded in an illustrative rather than a restrictive sense. The accompanying drawings that form a part hereof show, by way of illustration, and not of limitation, specific embodiments in which the subject matter may be practiced. The embodiments illustrated are described in sufficient detail to enable those, skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or an are used, as is common in patent documents, to include one or more than one, independent of any other instances or usages of “at least one” or “one or more,” In this document, the term “or” is used to refer to a nonexclusive or, such that “A or B” includes “A but not B,” “B but not A,” and “A and B,” unless otherwise indicated, in this document, the terms “including” and “in which” are used as the plain-English equivalents of the respective terms “comprising” and “wherein.” Also, in the following claims, the terms “including” and “comprising” are open-ended, that is, a system, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. § 1.72(b), requiring an abstract that will allow the reader to quickly ascertain the nature of the technical disclosure, It is submitted with the understanding that it will not be used to interpret or limit the scope or meaning of the claims. In addition, in the foregoing Detailed. Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

1. A method comprising:
receiving sensor data representing airflow or air pressure;
determining, using an artificial neural network, a current sleep stage corresponding to the sensor data, wherein the current sleep stage is one of: wake, rapid eye movement (REM), light sleep, and deep sleep, wherein the artificial neural network comprises a convolutional neural network (CNN), a recurrent neural network (RNN), and a conditional random field (CRF); and
providing an output representing the current sleep stage.

2. The method of claim 1, wherein the sensor data is received from a sensor residing on a face mask of a person, wherein the airflow or the air pressure comprises an oral or a nasal airflow or air pressure of the person.

3. The method of claim 1, wherein the RNN comprises a plurality of gated recurrent units (GRUs).

4. The method of claim 3, wherein at least one GRU from the plurality of GRUs comprises an update gate and a hidden cell state, wherein the at least one GRU computes a set of update equations based on an input received at the update gate and the hidden cell state.

5. The method of claim 1, wherein the artificial neural network comprises the CNN followed by the RNN followed by the CRF, wherein the CRF generates the output representing the current sleep stage

6. The method of claim 1, wherein the CNN comprise a plurality of blocks, each block comprising a one-dimensional (1D) convolution, followed by a rectified linear unit (ReLU), followed by a dropout.

7. The method of claim 6, wherein, in at least one block from the plurality of blocks, the dropout is followed by a max-pooling.

8. The method of claim 1, wherein the current sleep stage is determined based, at least in part, on a past sleep stage.

9. The method of claim 1, further comprising:
transmitting, using a wired or wireless communication interface, a control signal based on the current sleep stage, the control signal to control a device proximate to a sensor from which the sensor data is received.

10. The method of claim 9, wherein the control signal is to control one or more of: an oxygen provision device, a facial pressure device, a lighting device, a heating, ventilation, and air conditioning (HVAC) device, a white noise device, and an alarm clock.

11. A non-transitory machine-readable medium storing instructions which, when executed by processing circuitry of one or more machines, cause the processing circuitry to perform operations comprising:
receiving sensor data representing airflow or air pressure;
determining, using an artificial neural network, a current sleep stage corresponding to the sensor data, wherein the current sleep stage is one of: wake, rapid eye movement (REM), light sleep, and deep sleep, wherein the artificial neural network comprises a convolutional neural network (CNN), a recurrent neural network (RNN), and a conditional random field (CRF); and
providing an output representing the current sleep stage.

12. The machine-readable medium of claim 11, wherein the sensor data is received from a sensor residing on a face mask of a person, wherein the airflow or the air pressure comprises an oral or a nasal airflow or air pressure of the person.

13. The machine-readable medium of claim 11 where the RNN comprises a plurality of gated recurrent units (GRUs).

14. The machine-readable medium of claim 13, wherein at least one GRU from the plurality of GRUs comprises an update gate and a hidden cell state, wherein the at least one GRU computes a set of update equations based on an input received at the update gate and the hidden cell state.

15. The machine-readable medium of claim 11, wherein the artificial neural network comprises the CNN followed by the RNN followed by the CRF, wherein the CRF generates the output representing the current sleep stage.

16. The machine-readable medium of claim 11, wherein the CNN comprise a plurality of blocks, each block comprising a one-dimensional (1D) convolution, followed by a rectified linear unit (ReLU) followed by a dropout.

17. An apparatus comprising:
a data receiver to receive sensor data representing airflow or air pressure;
a memory storing an artificial neural network, the artificial neural network to determine a current sleep stage corresponding to the sensor data, wherein the current sleep stage is one of: wake, rapid eye movement (REM), light sleep, and deep sleep, wherein the artificial neural network comprises a convolutional neural network (CNN), a recurrent neural network (RNN), and a conditional random field (CRF);
processing circuitry to execute the artificial neural network; and
an output device to provide an output representing the current sleep stage.

18. The apparatus of claim 17, wherein the data receiver comprises a wireless radio or a wired connection.

19. The apparatus of claim 17, wherein the output device comprises a network interface card or a display port.

20. The apparatus of claim 17, wherein the output device is further to transmit a control signal based on the current sleep stage, the control signal to control a device external to the apparatus.
